(19) United States
(12) Patent Application Publication
(10) Pub. No.: US 2007/0165837 A1
(43) Pub. Date: Jul. 19, 2007

(54) Synchronizing Input Streams for Acoustic Echo Cancellation

(75) Inventors: Wei Zhong, Issaquah, WA (US); Yong Xia, Beijing (CN)

Correspondence Address: LEE & HAYES PLLC, 421 W RIVERSIDE AVENUE SUITE 500, SPOKANE, WA, 99201, US

(73) Assignee: Microsoft Corporation, Redmond, WA (US)

(21) Appl. No.: 11/275,431

(22) Filed: Dec. 30, 2005

(51) Int. Cl. A61F 11/06; H04M 9/08; G10K 11/16; H03B 29/00

(52) U.S. Cl. 379/406.01; 381/71.1

(57) ABSTRACT

Input streams for acoustic echo cancellation are associated with timestamps using reference times from a common clock. A render delay occurs between when an inbound signal is written to a buffer and when it is retrieved for rendering. A capture delay occurs between when a capture signal is written to a buffer and when it is retrieved for transmission. Both the render delay and the capture delay are variable and independent of one another. A render timestamp applies the render delay as an offset to a reference time at which the inbound signal is written to the buffer for rendering. A capture timestamp applies the capture delay as an offset to a reference time at which when the capture signal is retrieved for transmission. Applying the delay times as offsets to the reference times from the common clock facilitates synchronizing the streams for echo cancellation.

### BACKGROUND

Voice Over Internet Protocol (VoIP) and other processes for communicating voice data over computing networks are becoming increasingly more widely used. VoIP, for example, allows households and businesses with broadband Internet access and a VoIP service to make and receive full duplex calls without paying for a telephone line, telephone service, or long distance charges.

In addition, VoIP software allows users to make calls using their computers' audio input and output systems without using a separate telephone device. As shown in FIG. 1, a user of a desktop computer 100 equipped with speakers 110 and a microphone 120 is able to use the desktop computer 100 as a hands-free speakerphone to make and receive telephone calls. Another person participating in the calls may use a telephone or a computer. The other user, for example, may use a portable computer 130 as a speakerphone, using speakers 140 and a microphone 150 integrated in the portable computer 130. Words spoken by the user of the desktop computer 100, represented as a first signal 160, are captured by the microphone 120 and carried via a network (not shown) to the portable computer 130, and sounds carried by the signal 160 are rendered by the integrated speakers 140. Similarly, words spoken by the user of the portable computer 130, represented as a second signal 170, are captured by the integrated microphone 150 and carried via the network to the desktop computer 100 and rendered by the speakers 110.

One problem encountered by VoIP users, particularly those who place calls using their computers' speakers and microphones instead of a headset, is acoustic echo, which is depicted in FIG. 2. Acoustic echo results when the words uttered by a first user, represented by a first audio signal 200, are rendered by the speakers 210 and then captured by the microphone 220 along with words spoken by a second user, represented by a second audio signal 230. The microphone 220 and supporting input systems (not shown) generate a combined signal 240 that includes some manifestation of the first audio signal 200 and the second audio signal 230. Thus, when the combined signal 240 is rendered for the first user, the first user will hear both what the second user said and an echo of what the first user previously said.

One solution to the echo problem employs acoustic echo cancellation (AEC). An AEC system monitors An AEC system monitors both signals captured from the microphone 220 and inbound signals representing sounds to be rendered. To cancel acoustic echo, the AEC system digitally subtracts the inbound signals that may be captured by the microphone 220 so that the person on the other end of the call will not hear an echo of what he or she said. The AEC system attempts to identify an echo delay between the rendering of the first audio signal by the speakers and the capture of the first audio signal by the microphone to digitally subtract the inbound signals from the combined signal at the correct point in time.

### SUMMARY

Input streams for acoustic echo cancellation are associated with timestamps using reference times from a common clock. A render delay occurs between when an inbound signal is written to a buffer and when it is retrieved for rendering. A capture delay occurs between when a capture signal is written to a buffer and when it is retrieved for transmission. Both the render delay and the capture delay are variable and independent of one another. A render timestamp applies the render delay as an offset to a reference time at which the inbound signal is written to the buffer for rendering. A capture timestamp applies the capture delay as an offset to a reference time at which when the capture signal is retrieved for transmission. Applying the delay times as offsets to the reference times from the common clock facilitates synchronizing the streams for echo cancellation.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

### BRIEF DESCRIPTION OF THE DRAWINGS

The detailed description is described with reference to the accompanying figures. In the figures, the left-most digit of a three-digit reference number or the two left-most digits of a four-digit reference number identifies the figure in which the reference number first appears. The use of the same reference numbers in different figures indicates similar or identical items.

FIG. 1 (Background) is a perspective diagram of two computing systems permitting users to engage in voice data communications.

FIG. 2 (Background) is a schematic diagram illustrating capture of a received sound resulting in acoustic echo.

FIG. 3 is a schematic diagram of a computing system using an acoustic echo cancellation (AEC) to attempt to suppress acoustic echo.

FIG. 4 is a flow diagram of a mode of associating rendered and captured signals with timestamps using a common clock to facilitate AEC.

FIG. 5 is a schematic diagram of a computing system using a mode of associating timestamps with rendered and captured signals.

FIG. 6 is a graphical representation of a mode of deriving a render timestamp for a rendered output.

FIG. 7 is a graphical representation of a mode of deriving a capture timestamp for a captured output.

FIGS. 8 and 9 are graphical representations of a mode of associated timestamps accounting for render delays in canceling acoustic echo.

FIG. 10 is a flow diagram of a mode of using timestamps from a reference clock to synchronize rendered and captured signals to facilitate AEC.

FIG. 11 is a block diagram of a computing-system environment suitable for deriving, associating, and using timestamps to facilitate AEC.

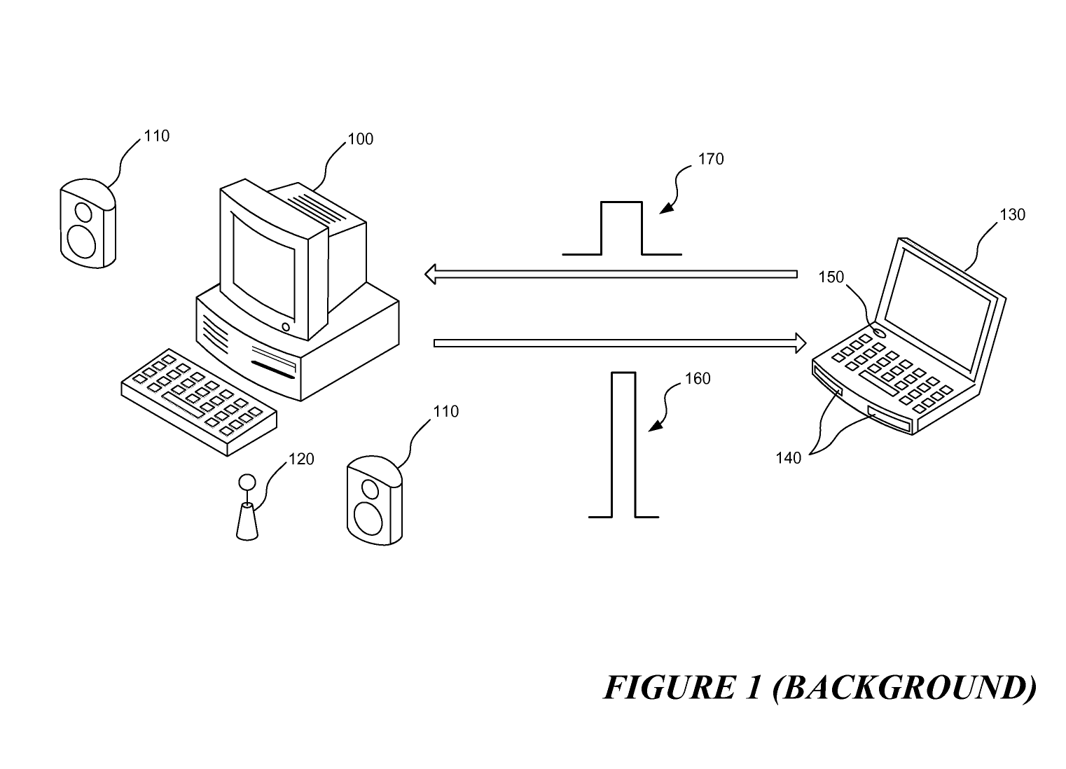

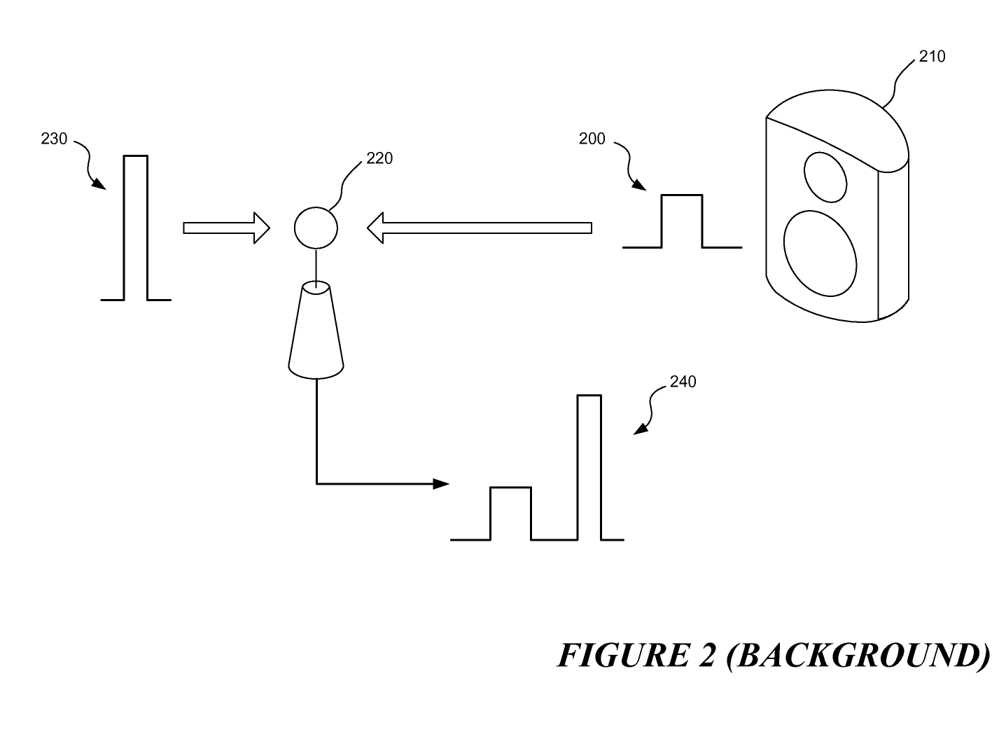

### DETAILED DESCRIPTION

Input streams for AEC are associated with timestamps based on a common reference clock. An inbound signal, from which audio will be rendered is associated with a timestamp, and a captured signal representing outbound audio, is associated with a timestamp. Because the timestamps use reference times from a common clock, variable delays resulting from processing of rendered signals and captured signals are reconciled relative to the common clock. Thus, the only variable in performing AEC is the echo delay between generation of sounds from the rendered signal and the capture of those sounds by a microphone. Associating the timestamps with the inbound signal and the captured signal facilitates AEC by eliminating delay variables for which AEC may be unable to account.

Variables in AEC

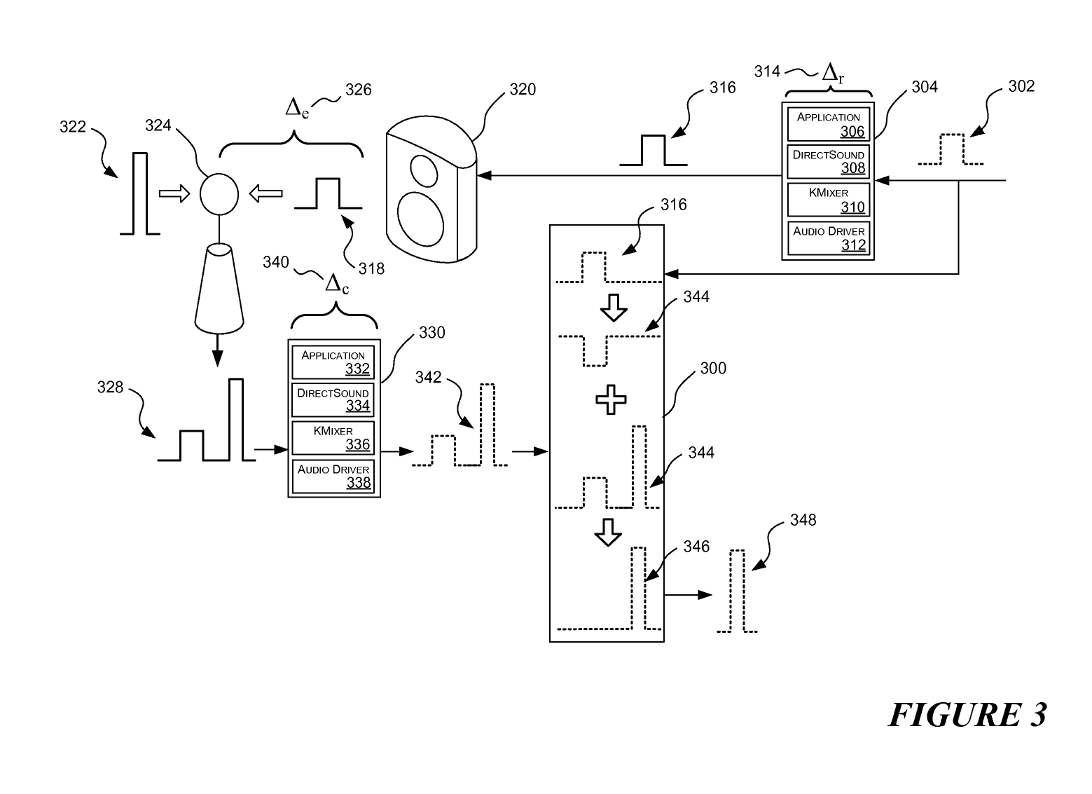

FIG. 3 illustrates a computing environment in which an AEC system 300 is used to remove or reduce acoustic echo. In FIG. 3, an inbound signal 302 represents words uttered by a caller (not shown). The signal 302 typically is presented in a series of frames, the size of which are determined by an audio codec (not shown) that retrieves the inbound signal 302 from inbound data.

The inbound signal 302 is received by a rendering system 304 executing in the computing system. The rendering system 304 includes a plurality of layers, including an application 306, such as a VoIP application, a sound module such as DirectSound module 308 used in Microsoft Windows®, a kernel audio mixer such as a KMixer 310 also used in Microsoft Windows®, and an audio driver 312 that supports the output hardware. Processing of threads in the layers 306-312 results in a render delay Δᵣ 314 between when data carrying the inbound signal 302 are written to a buffer in the DirectSound module 308 and when the data are read from the buffer to be rendered to produce a rendered output 316. Practically, the DirectSound module 308 “plays” the data from the buffer by reading the data from the buffer and presenting it to the audio driver 312. The rendered output 316 is presented to audio hardware to produce a rendered sound 318. In FIG. 3, the audio hardware is represented by a speaker 320, although it should be appreciated that other hardware, such as a sound card, amplifier, or other audio hardware (not shown), frequently is involved in generating the rendered sound 318.

In addition to being input to the rendering system 304, the inbound signal 302 also is input to the AEC system 300. As further described below, the AEC system 300 attempts to cancel acoustic echo by removing the inbound signal 302 from outbound transmissions.

The rendered sound 318 produced by the speaker 320 and a local sound 322, such as words spoken by a local user (not shown), are captured by a microphone 324. The rendered sound 318 reaches the microphone 324 after an echo delay Δₑ 326. The echo delay Δₑ 326 includes a propagation delay between the time the rendered sound 318 is generated by the speaker 320 and captured by the microphone 324. The echo delay Δₑ 326 also includes any other delay that may occur from the time the rendering system 304 generates the rendered output 316 and the time the capture system 330 logs the composite signal 328. The AEC system 300 identifies the echo delay Δₑ 326 to cancel the echo resulting from the rendered sound 318.

A composite signal 328 captured by the microphone 324 includes both the local sound 322 and some manifestation of the rendered sound 318. The manifestation of the rendered sound 318 may be transformed by gain or decay resulting from the audio hardware, multiple audio paths caused by reflected sounds, and other factors. The composite signal 328 is processed by a capture system 330 which, like the rendering system 304, includes a plurality of layers, including an application 332, a sound module such as DirectSound module 334, a kernel audio mixer such as a KMixer 336, and an audio driver 338 that supports the input hardware. In a mirror image of rendering system 304, there is a capture delay Δ_{c} 340 between a time when data carrying the composite signal 328 are captured by the audio driver 338 and are read by the application 332 and processed by the KMixer 336 and the audio driver 338. The captured output 342 of the capture system 330 is presented to the AEC system 300.

The AEC system 300 attempts to cancel acoustic echo by digitally subtracting a manifestation of the inbound signal 302 from the captured output 342. This is represented in FIG. 3 as an inverse 344 of the inbound signal 302 being added to the captured output 342 to yield a corrected signal 346. Ideally, the corrected signal 346 represents the local sound 322 without the echo resulting from repeating the rendered sound 318 being captured by the microphone 324. The corrected signal 346 is presented as the output 348 of the AEC system 300.

The AEC system 300 attempts to isolate the echo delay Δₑ 326 to synchronize the captured output 342 with the inbound signal 302 to cancel the inbound signal 302. However, if the inbound signal 302 is not subtracted from the captured output 342 at the point in time where the inbound signal 302 was maniested as the rendered output 316 and captured by the microphone 324, the echo will not be cancelled. Moreover subtracting the inbound signal 302 from the captured output 342 at the wrong point may distort the local sound 320 in the output 348 of the AEC system 300.

Associating Timestamps with Render and Capture Signals

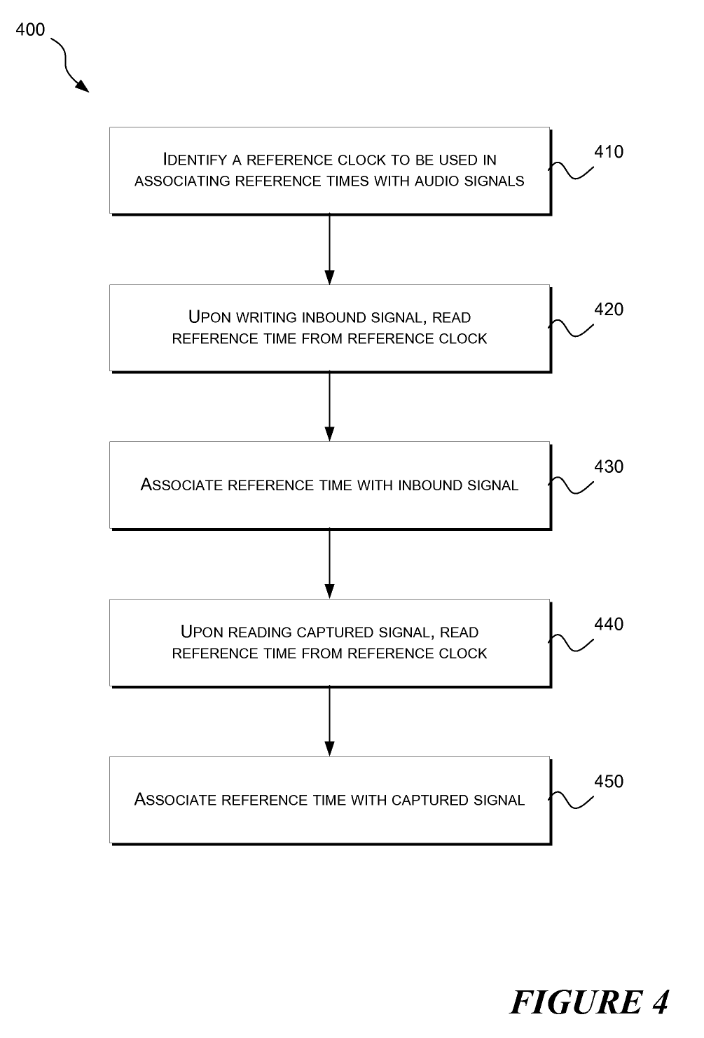

FIG. 4 is a flow diagram of a process 400 of associating timestamps with inbound and captured signals. At 410, a reference clock or “wall clock” is identified that will be used in generating the timestamps to be associated with the inbound and captured signals. The reference clock may be any clock to which both the render and capture systems have access. In one mode, the reference clock may be a system clock of a computing system supporting the audio systems performing the render and capture operations. Alternatively, for example, a reference clock may be a subsystem clock maintained by an audio controller or another system.

At 420, upon the inbound signal being written to a buffer, such as an application writing the inbound signal to a DirectSound buffer as previously described, a reference time is read from the reference clock. At 430, the reference time is associated with the inbound signal. As will be further described below, in systems where there is a variable render delay between when the inbound signal is written to the buffer and retrieved for rendering, the render delay is added or otherwise applied to the reference time to create a timestamp that allows for the synchronization of the inbound signal and the captured signal to facilitate AEC. Alternatively, in a system where the captured delay is minimal or nonvariable, a timestamp including only the reference time still may be used by an AEC system in order to help identify an acoustic echo interval.

At 440, upon the captured signal being read from a buffer, such as by an application from a DirectSound buffer, another reference time is read from the reference clock. At 450, the reference time is associated with the captured signal. Again, the reference time may be offset by a capture delay or otherwise used to help identify an echo interval, as further described below.

System for Associating Delay-Adjusted Timestamps with Signals

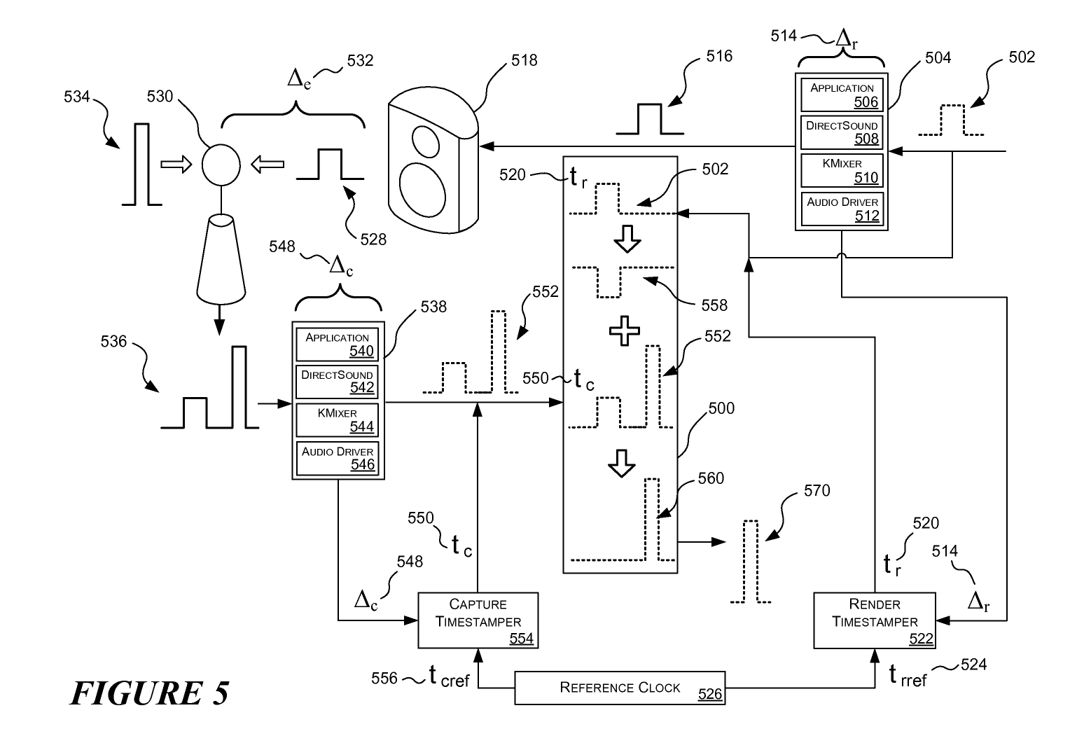

FIG. 5 is a block diagram of an exemplary system that might be used in VoIP communications or other applications where acoustic echo may present a concern. FIG. 5 shows an embodiment of a system in which timestamps are associated with render and capture signals. In the embodiment shown in FIG. 5, the timestamps are based on reference times from a reference clock that are combined with render and capture delay times.

FIG. 5 shows a computing system including an AEC system 500 to cancel acoustic echo. In the example of FIG. 5, an inbound signal 502 represents words spoken by a caller and received over a network. The inbound signal 502 is submitted to the AEC system 500 and to a rendering system 504.

As previously described, the rendering system 504 includes a plurality of layers including an application 506, a DirectSound module 508, a KMixer 510, and an audio driver 512. The computing system's processing of threads within the layers 506-512 and in other programs executing on the computing system results in a render delay Δᵣ 514. In one mode, the render delay Δᵣ 514 is an interval between when data carrying the signal 502 are written by the application 506 to a buffer in the DirectSound module 508 and when the data carrying the signal 502 are read from the buffer to be rendered. After the passing of the render delay Δᵣ 514, a rendered output 516 is presented both to the audio hardware 520 and the AEC system 500.

The render delay Δᵣ 514 can be identified by the application. For example, an application program interface (API) supported by the DirectSound module 508 supports API calls that allow the application 506 to determine or estimate how long it will be before frames being written to the DirectSound buffer will be retrieved for rendering. The interval may be derived by retrieving a current time representing when frames are being written to the buffer and a time at which frames currently being retrieved for rendering were written to the buffer. The render delay Δᵣ 514 is the difference between these two times.

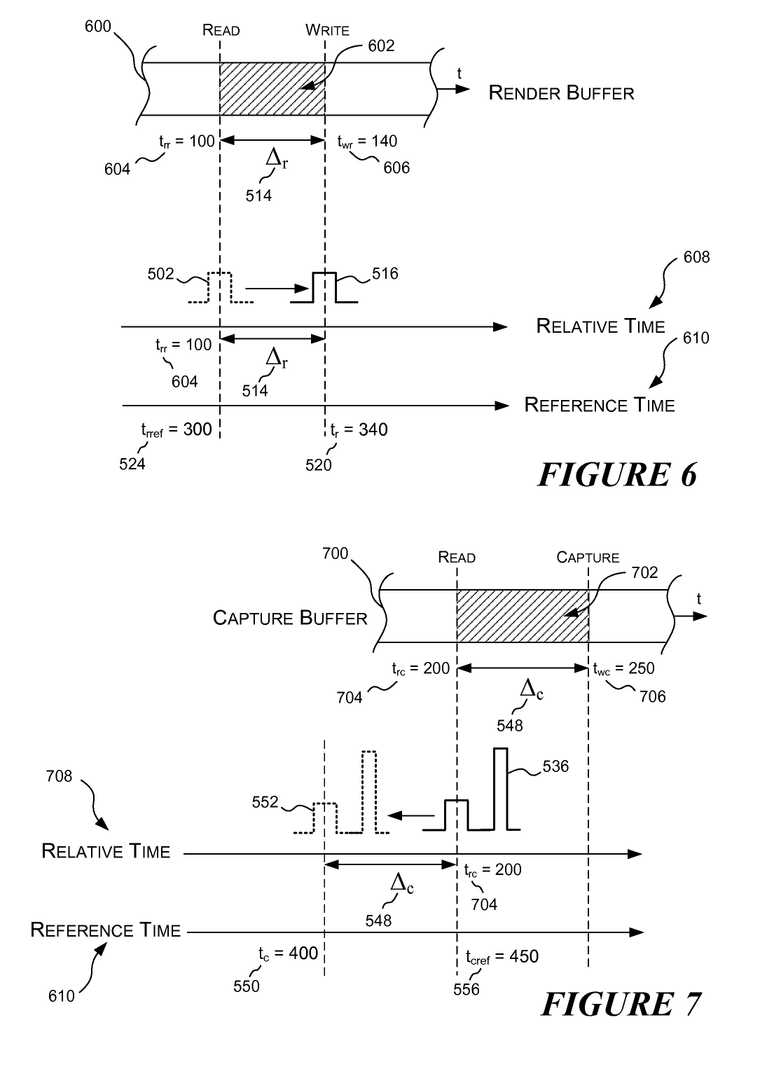

For illustration, FIG. 6 represents a render buffer 600 in which audio data 602 have been written and from which audio data 602 are currently being read for rendering. In the example of FIG. 6, data 602 currently being read for rendering was written at a time of tᵣᵣ 604 of 100 milliseconds, while audio data 602 are currently being written for subsequent rendering at time t_{wr} 606 of 140 milliseconds. Times tᵣᵣ 604 and t_{wr} 606 are expressed in a relative time 608 recognized by a module, such as a DirectSound module, maintaining the buffer 600. Thus, in this example, the render delay Δᵣ 514 is 40 milliseconds between when audio data 602 currently are written to the buffer 600 and currently are being read from the buffer 600. An API may directly provide the net difference, which is the render delay Δᵣ 514, or the API may provide the times tᵣᵣ 604 and t_{wr} 606 from which the net difference representing the render delay Δᵣ 514 is determined.

An effect of the render delay Δᵣ 514 can also is shown in FIG. 6. For the sake of example, the data written at tᵣᵣ 604 that currently is being read are assumed to be the data representing the inbound signal 502. It is further assumed that data representing the inbound signal 502 was written to the buffer 600 at time tᵣᵣ 604 of 100 milliseconds in a relative time 608 recognized by the rendering system. At the same time the data written at tᵣᵣ 604 is being read, new data currently is being written at time t_{wr} 606, which is assumed to be 140 milliseconds. Thus, it is estimated that data currently being written at t_{wr} 606 will be read after the passing of the 40 millisecond render delay Δᵣ delay 514.

Three aspects of the example of FIG. 6 should be noted. First, the write and read times provided by the API calls are based on a relative time 608 and do not correspond to a system time or other standard time. Second, while the timestamps are provided in units of time, the timestamps may be presented in terms of quantities of data instead of time. Given a known sampling rate, such as a number of samples taken per second, and a quantization value expressing the number of bytes per sample, a timestamp expressed in terms of a quantity of data translates directly to a measure of time. Third, the render delay Δᵣ 514 derived from the API calls actually is an estimate of when data currently being written to the buffer will be rendered, based on how far in advance data are being read in advance of data currently being written. Nonetheless, a render delay Δᵣ 514 determined by this estimate provides an indication of when data currently being written to the buffer will be read for rendering for use in creating an appropriate timestamp.

Referring again to the embodiment of FIG. 5, a render delay Δᵣ 514 is used in generating a render timestamp tᵣ 520 that is associated with the inbound signal 502. A render timestamper 522 receives both the render delay Δᵣ 514 and a render reference time t_{rref} 524 that is read from a reference clock 526. As previously described, the reference clock 526 may be a system clock or other clock accessible both to the rendering system and the capture system to provide a source of reference times that can be used by the AEC system 500 to synchronize the input streams.

In one mode, when data representing the inbound signal 502 are written to the buffer, the render timestamper 522 reads the current time presented by the reference clock 526 as the render reference time t_{rref} 524. The render timestamper 522 also reads the render delay Δᵣ 514 at the same time, or as nearly as possible to the same time, the data representing the inbound signal 502 are written. The render timestamper 522 adds the render reference time t_{rref} 524 to the render delay Δᵣ 514 to generate the render timestamp tᵣ 520 according to Eq. (1):
tᵣ=t_{rref}+Δᵣ (1)
The render timestamp tᵣ 520 is associated with the inbound signal 502. The render timestamp tᵣ 520 indicates to the AEC system 500 when the inbound signal 502 will be read and presented as the rendered output 516 and applied to the audio hardware 518. Thus, the render timestamp tᵣ 520, relative to the time maintained by the reference clock 526, indicates when the inbound signal 502 will result in generation of an output sound 528 that may produce an undesirable acoustic echo.

For illustration, referring again to FIG. 6, the render delay Δᵣ 514 was determined to be 40 milliseconds when the data representing the inbound signal 502 were read at tᵣᵣ 60r. As described with regard to FIG. 5, at tᵣᵣ 604, a render reference time t_{rref} 524 is read from a system clock or other reference clock that is recognized as the source of a reference time 610 that will be used both in generating render and capture timestamps. For sake of a numeric example, when the data representing the inbound signal 502 written at tᵣᵣ 604 are read, it is assumed the render reference time t_{rref} 524 is 300 milliseconds. According to Eq. (1), a render timestamp tᵣ 520 is equal to the sum of the render reference time t_{rref} 524, 300 milliseconds, and the render delay Δᵣ 514, 40 milliseconds, resulting in a render timestamp tᵣ 520 of 340 milliseconds. The use of the render timestamp tᵣ 520 in facilitating AEC is described further below.

Referring again to FIG. 5, the output sound 528 will reach a microphone 530 after an echo delay Δₑ 532. The microphone 530 also will capture local sounds 534 such as words spoken by a user. Thus, the microphone 530 and other input hardware will generate a composite signal 536 that potentially includes both the local sounds 534 and an echo resulting from the output sound 528. The composite signal 536 is submitted to a capture system 538. As in the case of the rendering system 504, the capture system 538 includes an application 540, a DirectSound module 542, a KMixer 544, and an audio driver 546 that supports the input hardware. For the sake of clarification, the capture system 538 and its layers 540-546 are represented separately from the rendering system 504 and its layers 506-512 even though the capture system 538 and the rendering system 504 may be supported by the same or corresponding instances of the same modules.

In a mirror image of the process by which signals are processed by the rendering system 504, in the capture system 538 there is a capture delay Δ_{c} 548 between a time when data representing the composite signal 536 are captured by the audio driver 546 and written to a buffer in the DirectSound module 542 and when the application 540 reads the frames for transmission or other processing. The resulting expected capture delay Δ_{c} 548 is illustrated in FIG. 7.

FIG. 7 shows a capture buffer 700 into which captured data 702 have been written and from which captured data 702 are being read. In the example of FIG. 7, captured data 702 currently being read for transmission or processing were captured at time t_{rc} 704 of 200 milliseconds while data are currently being captured to the capture buffer 700 at a time of t_{cc} 706 of 250 milliseconds. Thus, in this example, the capture delay Δ_{c} 548 between when data are being written to the capture buffer 700 and are being read from the capture buffer 700 is 50 milliseconds. Times t_{rc} 704 and t_{cc} 706 are based on a relative time 708 provided by the module maintaining the capture buffer 700.

An effect of the capture delay Δ_{c} 548 is that data 702 representing captured audio, such as the composite signal 536, currently written to the capture buffer 700 at time t_{wc 7}06 will be retrieved from the capture buffer 700 as rendered as a captured output 552 after a capture delay Δ_{c} 548 of 50 milliseconds. In other words, data read at time t_{rc} 704 represents sounds written to the capture buffer 700 at point 50 milliseconds earlier. Comparable to the case of the render buffer 600 (FIG. 6), the capture delay Δ_{c} 548 derived from the API calls actually is an estimate of when data currently being read from the buffer were written to the buffer, based on how far in advance data currently are being written to the buffer in advance of data currently being read.

Referring again to FIG. 5, in one mode, the expected capture delay Δ_{c} 548 is used in generating a capture timestamp t_{c} 550 that is associated with the captured output 552. A capture timestamper 554 receives both the capture delay Δ_{c} 548 and a capture reference time t_{cref} 556 that is read from the reference clock 526.

In one mode, when data representing the composite signal 536 are being read from the buffer to generate the captured output 552, the capture timestamper 554 reads the current time presented by the reference clock 526 as the capture reference time t_{cref} 556. The capture timestamper 554 also reads the capture delay Δ_{c} 548 at the same time, or as nearly as possible to the same time, the data representing the captured output 552 are being read. In contrast to the render timestamper 552, however, the capture timestamper 554 subtracts the capture delay Δ_{c} 548 from the capture reference time t_{cref} 556 to generate the render timestamp t_{c} 550 according to Eq. (2):
t_{c}=t_{cref}−Δ_{c} (2)
The capture timestamp t_{c} 550 is associated with the captured output 552. The capture timestamp t_{c} 550 indicates to the AEC system 500 when the composite signal 536 represented by the captured output 552 was captured by the microphone 530.

For illustration, referring again to FIG. 7, the capture delay Δ_{c} 548 was determined to be 50 milliseconds when the data representing the composite signal 536 are read at t_{rc} 704 to produce the captured output 552. As described with regard to FIG. 5, at t_{rc} 704 a capture reference time t_{cref} 556 is read from a system clock or other reference clock that is recognized as the source of the reference time 610 used both in generating render and capture timestamps. For sake of a numeric example, when the data representing the composite signal 536 are read at t_{rc} 704, it is assumed the capture reference time t_{cref} 556 is 450 milliseconds. According to Eq. (2), a capture timestamp t_{c} 550 is equal to the difference of the capture reference time t_{cref} 556, 450 milliseconds, and the capture delay Δ_{c} 548, 50 milliseconds, resulting in a capture timestamp t_{c} 550 of 400 milliseconds. The use of the capture timestamp t_{c} 550 in facilitating AEC is described further below.

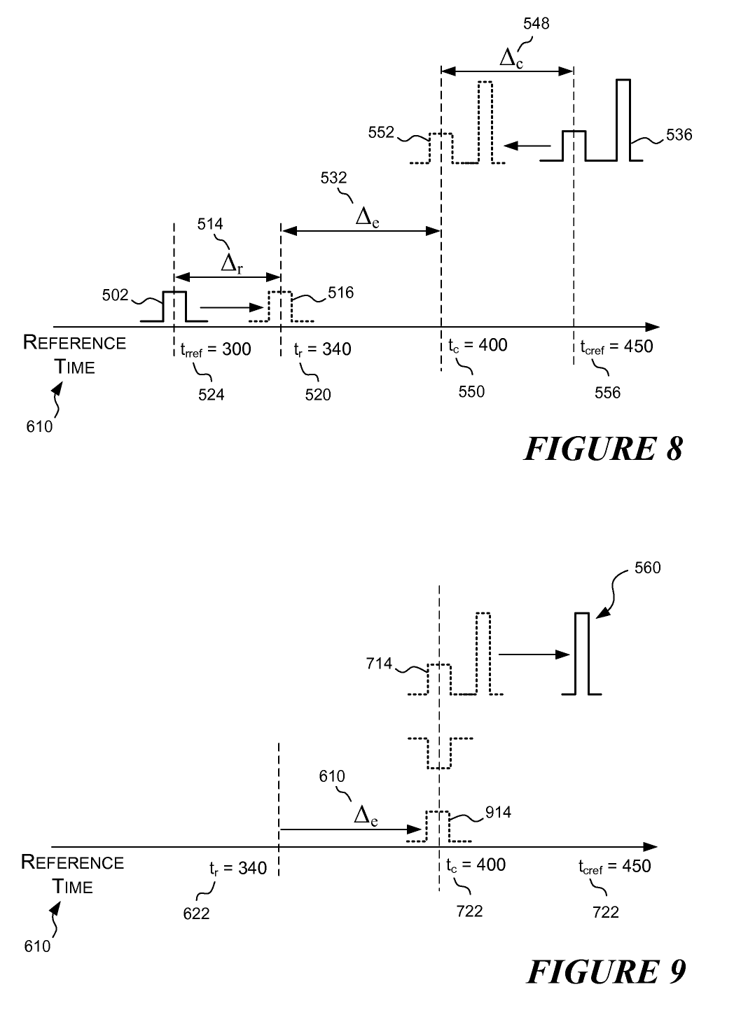

Referring again to FIG. 5, a conventional AEC system 500 is able to isolate the echo delay Δₑ 532 between generation of the output sound 528 and its receipt by the microphone 530 to facilitate removing the echo caused by the audio output 528 in the composite signal 536. A conventional AEC system may be able to identify the echo delay Δₑ 532 when the length of the echo delay Δₑ 532 is the only independent variable for which it must account. Therefore, it may be problematic or impossible for a conventional AEC system to isolate the echo delay Δₑ 532 when the render delay Δᵣ 514 and/or the capture delay Δ_{c} 548 vary. However, associating the render timestamp tᵣ 520 and the capture timestamp t_{c} 550 with the inbound signal 520 and the captured output 552, respectively, offsets variations in the render delay Δ_{c} 514 and the capture delay Δ_{c} 548, as illustrated in FIGS. 8 and 9. Furthermore, in a conventional AEC system, a search window in which the AEC system attempts to identify the echo delay Δₑ 532 may be shorter in duration than a total delay resulting from the render delay and the capture delay Δ_{c} 548. Although the search window may be increased to attempt to identify the echo delay Δₑ 532, increasing the search window introduces latency in the application for which echo cancellation is desired. Associating timestamps tᵣ 520 and t_{c} 550 with the signals therefore assists the AEC system in identifying the echo delay Δe 532 without introducing undesired latency.

FIG. 8 graphically illustrates relative displacement of the inbound signal 502 and the composite signal 536 offset by the render delay Δᵣ 514, the capture delay Δ_{c} 548, and the echo delay Δₑ 532. Data representing the inbound signal 502 are read to be presented as the rendered output 516 after a render delay Δᵣ 514. The render timestamp tᵣ 520 in the common reference time 610 provided by the reference clock 526 (FIG. 5) is 340 milliseconds. The render timestamp tᵣ 520 is equal to the sum of the render reference time t_{rref} 524 and the render delay Δᵣ 514. The data representing the composite signal 536 are read to be presented as the captured output 552 after a capture delay Δ_{c} 548. The capture timestamp t_{c} 550 in the common reference time 610 is 400 milliseconds. The capture timestamp t_{c} 550 is equal to the difference of the capture reference time t_{cref} 556 less the capture delay Δ_{c} 548. Thus, as shown in FIG. 8, the difference between the render timestamp tᵣ 520 and the capture timestamp t_{c} 550 is the same as the echo delay Δₑ 532. It should be appreciated that, because the speed of sound is approximately 340 meters per second, the echo delay Δₑ 532 depicted in the example of FIG. 8 is larger than may be anticipated in a typical setting. The echo delay Δₑ 532 is selected for clarity of illustration.

As shown in FIG. 9, by offsetting the rendered output 516 from the render timestamp tᵣ 520 of 340 milliseconds by the echo delay Δₑ 532, the rendered output 516 is situated opposite the captured output 552. Thus, an inverse 558 of the rendered output 516 can be applied to the captured output 552 to cancel the acoustic echo caused by the rendered output 516, producing a corrected signal 560 that yields the AEC output 570.

Using Timestamps to Facilitate AEC

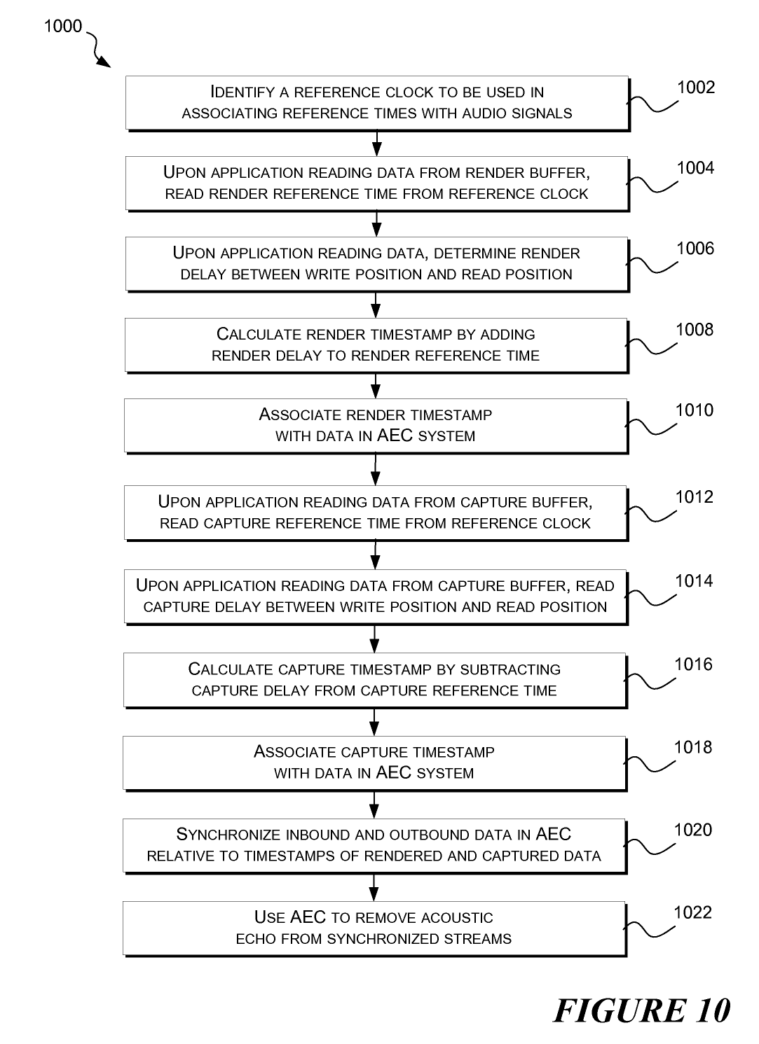

FIG. 10 is a flow diagram of a process 1000 using render and capture timestamps to facilitate AEC. At 1002, a reference clock or “wall clock” is identified that will be used in generating the timestamps to be associated with the inbound and captured signals. As previously described, the reference clock may be any clock to which both the render and capture systems have access. In one mode, the reference clock may be a system clock of a computing system supporting the audio systems performing the render and capture operations. Alternatively, for example, a reference clock may be a subsystem clock maintained by an audio controller or another system.

At 1004, upon an application, such as a VoIP application, reading data from a render buffer used to store inbound signals, a render reference time is read from a reference clock. At 1006, at the same time or as close as possible to the same time upon reading the data, the render delay is determined. As previously described, the render delay is the current delay between the current read time and the current write time, which can be determined from an API to the module supporting the render buffer. At 1008, the render timestamp is determined by adding the render delay to the render reference time. At 1010, the render timestamp is associated with the corresponding data in the AEC system.

At 1012, upon the application reading data from a capture buffer used to store outbound signals, a capture reference time is read from the reference clock. At 1014, at the same time or as close as possible to the same time upon reading the data, the capture delay is determined. Again, the capture delay is the current delay between the current read time from the capture buffer and the current write time to the capture, which can be determined from an API to the module supporting the buffer. At 1016, the capture timestamp is determined by subtracting the capture delay from the capture reference time. At 1018, the capture timestamp is associated with the corresponding data in the AEC system.

At 1020, the inbound and outbound data are synchronized in the AEC system using the timestamps to isolate the echo delay, as described with reference to FIGS. 8 and 9. At 1022, AEC is used to remove acoustic echo resulting from the inbound data from the outbound in the synchronized streams.

Computing System for Implementing Exemplary Embodiments

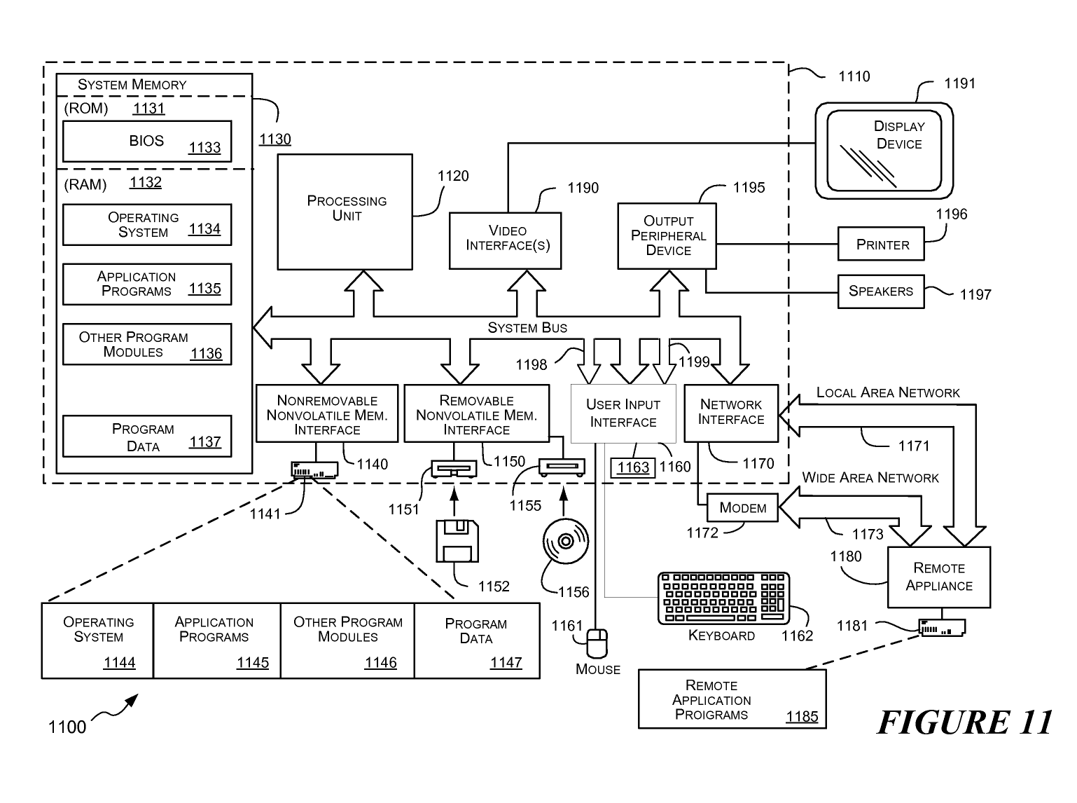

FIG. 11 illustrates an exemplary computing system 1100 for implementing embodiments of deriving, associating, and using timestamps to facilitate AEC. The computing system 1100 is only one example of a suitable operating environment and is not intended to suggest any limitation as to the scope of use or functionality of exemplary embodiments of deriving, associating, and using timestamps to facilitate AEC as previously described, or other embodiments. Neither should the computing system 1100 be interpreted as having any dependency or requirement relating to any one or combination of components illustrated in the exemplary computing system 1100.

Processes of deriving, associating, and using timestamps to facilitate AEC may be described in the general context of computer-executable instructions, such as program modules, being executed on computing system 1100. Generally, program modules include routines, programs, objects, components, data structures, etc., that perform particular tasks or implement particular abstract data types. Moreover, those skilled in the art will appreciate that processes of deriving, associating, and using timestamps to facilitate AEC may be practiced with a variety of computer-system configurations, including hand-held devices, multiprocessor systems, microprocessor-based or programmable-consumer electronics, minicomputers, mainframe computers, and the like. Processes of deriving, associating, and using timestamps to facilitate AEC may also be practiced in distributed-computing environments where tasks are performed by remote-processing devices that are linked through a communications network. In a distributed-computing environment, program modules may be located in both local and remote computer-storage media including memory-storage devices.

With reference to FIG. 11, an exemplary computing system 1100 for implementing processes of deriving, associating, and using timestamps to facilitate AEC includes a computer 1110 including a processing unit 1120, a system memory 1130, and a system bus 1121 that couples various system components including the system memory 1130 to the processing unit 1120.

The computer 1110 typically includes a variety of computer-readable media. By way of example, and not limitation, computer-readable media may comprise computer-storage media and communication media. Examples of computer-storage media include, but are not limited to, Random Access Memory (RAM); Read Only Memory (ROM); Electronically Erasable Programmable Read Only Memory (EEPROM); flash memory or other memory technology; CD ROM, digital versatile discs (DVD) or other optical or holographic disc storage; magnetic cassettes, magnetic tape, magnetic disk storage or other magnetic storage devices; or any other medium that can be used to store desired information and be accessed by computer 1110. The system memory 1130 includes computer-storage media in the form of volatile and/or nonvolatile memory such as ROM 1131 and RAM 1132. A Basic Input/Output System 1133 (BIOS), containing the basic routines that help to transfer information between elements within computer 1110 (such as during start-up) is typically stored in ROM 1131. RAM 1132 typically contains data and/or program modules that are immediately accessible to and/or presently being operated on by processing unit 1120. By way of example, and not limitation, FIG. 11 illustrates operating system 1134, application programs 1135, other program modules 1136, and program data 1137.

The computer 1110 may also include other removable/nonremovable, volatile/nonvolatile computer-storage media. By way of example only, FIG. 11 illustrates a hard disk drive 1141 that reads from or writes to nonremovable, nonvolatile magnetic media, a magnetic disk drive 1151 that reads from or writes to a removable, nonvolatile magnetic disk 1152, and an optical-disc drive 1155 that reads from or writes to a removable, nonvolatile optical disc 1156 such as a CD-ROM or other optical media. Other removable/nonremovable, volatile/nonvolatile computer-storage media that can be used in the exemplary operating environment include, but are not limited to, magnetic tape cassettes, flash memory units, digital versatile discs, digital video tape, solid state RAM, solid state ROM, and the like. The hard disk drive 1141 is typically connected to the system bus 1121 through a nonremovable memory interface such as interface 1140. Magnetic disk drive 1151 and optical dick drive 1155 are typically connected to the system bus 1121 by a removable memory interface, such as interface 1150.

The drives and their associated computer-storage media discussed above and illustrated in FIG. 11 provide storage of computer-readable instructions, data structures, program modules and other data for computer 1110. For example, hard disk drive 1141 is illustrated as storing operating system 1144, application programs 1145, other program modules 1146, and program data 1147. Note that these components can either be the same as or different from operating system 1134, application programs 1135, other program modules 1136, and program data 1137. Typically, the operating system, application programs, and the like that are stored in RAM are portions of the corresponding systems, programs, or data read from hard disk drive 1141, the portions varying in size and scope depending on the functions desired. Operating system 1144, application programs 1145, other program modules 1146, and program data 1147 are given different numbers here to illustrate that, at a minimum, they can be different copies. A user may enter commands and information into the computer 1110 through input devices such as a keyboard 1162; pointing device 1161, commonly referred to as a mouse, trackball or touch pad; a wireless-input-reception component 1163; or a wireless source such as a remote control. Other input devices (not shown) may include a microphone, joystick, game pad, satellite dish, scanner, or the like. These and other input devices are often connected to the processing unit 1120 through a user-input interface 1160 that is coupled to the system bus 1121 but may be connected by other interface and bus structures, such as a parallel port, game port, IEEE 1194 port, or a universal serial bus (USB) 1198, or infrared (IR) bus 1199. As previously mentioned, input/output functions can be facilitated in a distributed manner via a communications network.

A display device 1191 is also connected to the system bus 1121 via an interface, such as a video interface 1190. Display device 1191 can be any device to display the output of computer 1110 not limited to a monitor, an LCD screen, a TFT screen, a flat-panel display, a conventional television, or screen projector. In addition to the display device 1191, computers may also include other peripheral output devices such as speakers 1197 and printer 1196, which may be connected through an output peripheral interface 1195.

The computer 1110 will operate in a networked environment using logical connections to one or more remote computers, such as a remote computer 1180. The remote computer 1180 may be a personal computer, and typically includes many or all of the elements described above relative to the computer 1110, although only a memory storage device 1181 has been illustrated in FIG. 11. The logical connections depicted in FIG. 11 include a local-area network (LAN) 1171 and a wide-area network (WAN) 1173 but may also include other networks, such as connections to a metropolitan-area network (MAN), intranet, or the Internet.

When used in a LAN networking environment, the computer 1110 is connected to the LAN 1171 through a network interface or adapter 1170. When used in a WAN networking environment, the computer 1110 typically includes a modem 1172 or other means for establishing communications over the WAN 1173, such as the Internet. The modem 1172, which may be internal or external, may be connected to the system bus 1121 via the network interface 1170, or other appropriate mechanism. Modem 1172 could be a cable modem, DSL modem, or other broadband device. In a networked environment, program modules depicted relative to the computer 1110, or portions thereof, may be stored in the remote memory storage device. By way of example, and not limitation, FIG. 11 illustrates remote application programs 1185 as residing on memory device 1181. It will be appreciated that the network connections shown are exemplary, and other means of establishing a communications link between the computers may be used.

Although many other internal components of the computer 1110 are not shown, those of ordinary skill in the art will appreciate that such components and the interconnections are well-known. For example, including various expansion cards such as television-tuner cards and network-interface cards within a computer 1110 is conventional. Accordingly, additional details concerning the internal construction of the computer 1110 need not be disclosed in describing exemplary embodiments of processes of deriving, associating, and using timestamps to facilitate AEC.

When the computer 1110 is turned on or reset, the BIOS 1133, which is stored in ROM 1131, instructs the processing unit 1120 to load the operating system, or necessary portion thereof, from the hard disk drive 1141 into the RAM 1132. Once the copied portion of the operating system, designated as operating system 1144, is loaded into RAM 1132, the processing unit 1120 executes the operating system code and causes the visual elements associated with the user interface of the operating system 1134 to be displayed on the display device 1191. Typically, when an application program 1145 is opened by a user, the program code and relevant data are read from the hard disk drive 1141 and the necessary portions are copied into RAM 1132, the copied portion represented herein by reference numeral 1135.

Conclusion

Modes of synchronizing input streams to an AEC system facilitate consistent AEC. Associating the streams with timestamps from a common reference clock reconciles varying delays in rendering or capturing of audio signals. Accounting for these delays leaves the acoustic echo delay as the only variable for which the AEC system must account in cancelling undesired echo.

Although exemplary embodiments have been described in language specific to structural features and/or methodological acts, it is to be understood that the appended claims are not necessarily limited to the specific features or acts previously described. Rather, the specific features and acts are disclosed as exemplary embodiments.

1. A method comprising:
reading a first reference time from a reference clock upon writing a first signal to a rendering system;
associating with the first signal a first time derived at least in part from the first reference time;
reading a second reference time from the reference clock upon retrieving a second signal from a capture system; and
associating with the second signal a second time derived at least in part from the second reference time.

2. A method of claim 1, wherein the reference clock includes a system clock in a computing system supporting both the rendering system and the capture system.

3. A method of claim 1, further comprising deriving the first time by adjusting the first reference time by a first delay between when the first signal was received in the rendering system and when the first signal is retrieved from the rendering system.

4. A method of claim 3, wherein the first time is adjusted by adding the first delay to the first reference time.

5. A method of claim 1, further comprising deriving the second time by adjusting the second reference time by a second delay between when the second signal was received in the capture system and when the second signal is retrieved from the capture system.

6. A method of claim 5, wherein the second time is adjusted by subtracting the second delay from the second reference time.

7. A method of claim 1, further comprising correlating the first time and the second time to facilitate identifying whether the second signal was captured while a manifestation of the first signal was being presented.

8. A method of claim 7, further comprising at least partially removing the manifestation of the first signal from the second signal.

9. A method of claim 1, wherein the first signal is an inbound signal from a caller using a voice over Internet protocol application, and the second signal is an outbound signal directed to the caller.

10. A method, comprising:
receiving a render time associated with a rendered signal and derived at least in part from a first reference time read from a reference clock when the rendered signal is written to a render buffer storing the rendered signal;
receiving a capture time associated with a captured signal and derived at least in part from a second reference time read from the reference clock when the captured signal was read from a capture buffer storing the captured signal;
correlating the render time and the capture time to determine whether the captured signal at least partially includes the rendered signal.

11. A method of claim 10, wherein the reference clock includes a system clock in a computing system configured to process the rendered signal and the captured signal.

12. A method of claim 10, further comprising deriving the render time by adding the first reference time to a difference between when the rendered signal was received from a source by a rendering system and when the rendered signal is retrieved from the rendering system.

13. A method of claim 12, further comprising deriving the capture time by subtracting from the second reference time a difference between when the captured signal was acoustically received by the capture system and when the captured signal is retrieved from the capture system.

14. A method of claim 10, wherein correlating the render time and the capture time further comprises identifying an echo delay such that the echo delay accounts for a difference between the render time and the capture time.

15. A method of claim 14, further comprising, upon identifying that the captured signal includes a manifestation of the rendered signal, causing the manifestation of the rendered signal to be removed from the captured signal.

16. A timestamping system for assisting an echo cancellation system in synchronizing signals, comprising:
a reference time source; and
a time stamping system in communication with the reference time source and configured to provide to the echo cancellation system:
a render timestamp indicating a first reference time an inbound signal is provided to the echo cancellation system adjusted for a render delay in the inbound signal being rendered; and
a capture timestamp indicating a second reference time a captured signal is captured adjusted for a capture delay in the captured signal being presented to the echo cancellation system.

17. A system of claim 16, wherein the reference time source includes a system clock in a computing system configured to process the output signal and the input signal.

18. A system of claim 16, wherein:
the render delay includes a first interval between when the inbound signal is stored in a render buffer and is retrieved from the render buffer;
the capture delay includes a second interval between when the captured signal is stored in a capture buffer and is retrieved from the capture buffer.

19. A system of claim 16, wherein the render timestamp is adjusted by adding the render delay to the first reference time.

20. A system of claim 16, wherein the capture timestamp is adjusted by subtracting the capture delay from the second reference time.
